(19) United States
(12) Patent Application Publication
(10) Pub. No.: US 2018/0000107 A1
(43) Pub. Date: Jan. 4, 2018

(54) Herbal Tea Compositions for Babies

(71) Applicant: Samah Bensalem, Sunny Isles Beach, FL (US)

(72) Inventor: Samah Bensalem, Sunny Isles Beach, FL (US)

(21) Appl. No.: 15/703,219

(22) Filed: Sep. 13, 2017

Related U.S. Application Data
(63) Continuation-in-part of application No. 15/055,570, filed on Feb. 27, 2016.

(51) Int. Cl. A23F 3/00; A23L 33/105; A61K 36/28; A61K 36/235; A61K 9/00

(52) U.S. Cl. CPC A23F 3/00; A61K 36/235; A61K 9/0095; A61K 36/28; A23L 33/105; A23F 2200/00; A61K 2300/00

(57) ABSTRACT

Herbal tea compositions that, when consumed as a beverage by babies, eases common baby digestive discomforts and helps babies to sleep and feel better. The compositions comprise fennel seed, cumin seed. Some blends additionally contain dried chamomile flower.

### CROSS REFERENCE TO RELATED APPLICATION

This application is a continuation-in-part of prior U.S. patent application Ser. No. 15/055,570, filed Feb. 27, 2016, which application is hereby incorporated by reference.

### BACKGROUND

### 1. Technical Field

The subject matter described herein relates generally to herbal tea compositions, and in particular to herbal tea compositions which provide medicinal benefits when consumed.

### 2. Background Information

Babies often have digestive discomforts such as gas, constipation, acid reflux and colic which prevent them from sleeping for prolonged periods of time, and causes them to be irritable.

The present invention provides herbal tea compositions that, when consumed as a beverage by babies, eases common baby digestive discomforts and helps them sleep better and feel better.

### SUMMARY OF THE INVENTION

Herbal tea compositions according to the invention involve blends of fennel seed and cumin seed. Some blends may also include dried chamomile flower.

One of the herbal tea composition blends is made up of about 55% (by weight) to about 65% (by weight) fennel seed, with the remaining amount being cumin seed. A variation of this blend disclosed is more specifically defined where cumin seed makes up about 35% (by weight) to about 45% (by weight) of the composition.

Another herbal tea composition blend disclosed containing dried chamomile flower is made up of about 5% (by weight) to about 15% (by weight) of the composition, with the remaining amount being made up of fennel seed and cumin seed. A variation of this blend is more specifically defined where fennel seed makes up from about 45% (by weight) to about 55% (by weight) of the composition, cumin seed makes up from about 35% (by weight) to about 45% (by weight) of the composition, and dried chamomile flower makes up from about 5% (by weight) to about 15% (by weight) of the composition.

### DETAILED DESCRIPTION OF PREFERRED EMBODIMENTS

The disclosed subject matter now will be described more fully hereinafter with reference to the accompanying Examples, in which some, but not all embodiments of the disclosed subject matter are described. Detailed descriptions of preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the invention in virtually any appropriately detailed system, structure or manner.

Herbal tea compositions according to the invention involve blends of fennel seed and cumin seed. In preferred embodiments, the compositions can be prepared to comprise between about forty percent (40%) by weight to about seventy percent (70%) by weight fennel seed, and about thirty percent by weight (30%) to about sixty percent (60%) by weight cumin seed. Organically grown fennel and cumin seeds may be used in herbal tea compositions of the invention to provide herbal tea compositions that may be organically certified. A particularly desirable composition of the blend is about sixty percent (60%) by weight fennel seed and about forty percent (40%) by weight cumin seed. This particular composition has shown to be commercially desirable in that it provides the sought-after benefits of easing baby digestive discomforts, improving babies' sleep and mood, while presenting an herbal tea blend which is accepted by babies.

Another preferred herbal tea composition according to the invention involves blends of cumin seed, fennel seed and dried chamomile flower. In preferred embodiments, the compositions can be prepared to comprise between about thirty percent (30%) by weight to about sixty percent (60%) by weight fennel seed, about thirty percent (30%) by weight to about sixty percent (60%) by weight cumin seed, and about five percent (5%) by weight to about fifteen percent (15%) by weight dried chamomile flower. Dried chamomile flower made from organically grown chamomile flowers may be used in herbal tea compositions of the invention to provide herbal tea compositions that may be organically certified. A particularly desirable composition of the blend is about fifty percent (50%) by weight fennel seed, about forty percent (40%) by weight cumin seed, and about ten percent (10%) by weight dried chamomile flower. This particular composition has shown to be commercially desirable in that it provides the sought-after benefits of easing baby digestive discomforts, while increasing improvement in babies' sleep, and presenting an herbal tea blend with increased acceptance by babies.

### EXAMPLES

The following examples have been included to provide guidance to one of ordinary skill in the art for practicing representative embodiments of the disclosed subject matter.

### Example 1

### Representative Procedure for Preparing Herbal Tea Composition

### (60% Cumin Seed/40% Fennel Seed Blend)

The procedure described is for a three hundred (300) gram batch that produces approximately two hundred (200) tea bags.

Initially, one hundred eighty (180) grams cumin seed and one hundred twenty (120) grams fennel seed are selected and washed thoroughly. After the ingredients have air-dried in a sterilized manner, they are mixed thoroughly until a uniform consistency is achieved. The blend is then packaged in tea bags at one point five (1.5) grams per bag.

### Example 2

### Representative Procedure for Preparing Herbal Tea Composition

### (60% Fennel Seed/40% Cumin Seed Blend)

The procedure described is for a three hundred (300) gram batch that produces approximately two hundred (200) tea bags.

Initially, one hundred eighty (180) grams fennel seed and one hundred twenty (120) grams cumin seed are selected and washed thoroughly. After the ingredients have air-dried in a sterilized manner, they are mixed thoroughly until a uniform consistency is achieved. The blend is then packaged in tea bags at one point five (1.5) grams per bag.

### Example 3

### Representative Procedure for Preparing Herbal Tea Composition

### (50% Fennel Seed/40% Cumin Seed/10% Dried Chamomile Flower Blend)

The procedure described is for a three hundred (300) gram batch that produces approximately two hundred (200) tea bags.

Initially, one hundred fifty (150) grams fennel seed, one hundred twenty (120) grams cumin seed, and thirty (30) grams dried chamomile flower are selected and washed thoroughly. After the ingredients have air-dried in a sterilized manner, they are mixed thoroughly until a uniform consistency is achieved. The blend is then packaged in tea bags at one point five (1.5) grams per bag.

While the invention has been described in connection with preferred embodiments, they are not intended to limit the scope of invention to the particular forms set forth, but on the contrary, they are intended to cover such alternatives, modifications, and equivalents as may be included within the spirit and scope of the invention as defined by the appended claims.

What is claimed is:

1. An herbal tea composition, comprising:
a predetermined amount of fennel seeds; and
a predetermined amount of cumin seeds.

2. The composition of claim 1, wherein said predetermined amount of fennel seeds is from about 55% (by weight) to about 65% (by weight) of said composition.

3. The composition of claim 2, wherein said predetermined amount of cumin seeds is from about 35% (by weight) to about 45% (by weight) of said composition.

4. The composition of claim 1, further comprising a predetermined amount of dried chamomile flower.

5. The composition of claim 4, wherein said predetermined amount of dried chamomile flower is from about 5% (by weight) to about 15% (by weight) of said composition.

6. The composition of claim 4, wherein:
said predetermined amount of fennel seeds if from about 45% (by weight) to about 55% (by weight) of said composition;
said predetermined amount of cumin seeds is from about 35% (by weight) to about 45% (by weight) of said composition; and
said predetermined amount of dried chamomile flower is from about 5% (by weight) to about 15% (by weight) of said composition.
